(19) United States
(12) Patent Application Publication
(10) Pub. No.: US 2025/0361264 A1
(43) Pub. Date: Nov. 27, 2025

(54) PROTEIN MODIFYING REAGENT

(71) Applicants: CANON KABUSHIKI KAISHA, Tokyo (JP); CANON VIRGINIA, INC., Newport News, VA (US)

(72) Inventors: KEIGO MIZUSAWA, Tokyo (JP); YUMA KOBAYASHI, Kanagawa (JP); KEI INOUE, Kanagawa (JP); WAKA HASEGAWA, Tokyo (JP)

(21) Appl. No.: 19/297,520

(22) Filed: Aug. 12, 2025

Related U.S. Application Data
(63) Continuation of application No. PCT/JP2024/004846, filed on Feb. 13, 2024.

(30) Foreign Application Priority Data
Feb. 14, 2023 (JP) 2023-020824
Feb. 1, 2024 (JP) 2024-014494

(51) Int. Cl. C07K 1/08; C07K 16/00; C12N 9/00; C12N 15/11; G01N 33/68

(52) U.S. Cl. CPC C07K 1/08; C07K 16/00; C12N 9/00; C12N 15/11; G01N 33/68

(57) ABSTRACT

A purpose is to impart a function a protein having a β-sheet structure by using a material having a small molecular size. A modifying reagent for a protein having a β-sheet structure is provided, comprising a structure derived from a biomolecule and a group containing an aromatic ring, wherein the group containing the aromatic ring may has at least one selected from the group consisting of a benzothiazole group, a benzoxazole group, a benzimidazole group, and a naphthylazo group, each may have a substituent, the structure derived from the biomolecule includes a structure derived from one selected from a group consisting of an enzyme, an antibody, an antigen, a peptide, a polynucleotide, an oligonucleotide, a ligand, an enzyme substrate, a biotin, and a catecholamine.

### CROSS-REFERENCE TO RELATED APPLICATIONS

This application is a Continuation of International Patent Application No. PCT/JP2024/004846, filed Feb. 13, 2024, which claims the benefit of Japanese Patent Application No. 2023-020824, filed Feb. 14, 2023, and Japanese Patent Application No. 2024-014494, filed Feb. 1, 2024, all of which are hereby incorporated by reference herein in their entirety.

### BACKGROUND

### Field of the Technology

The present disclosure relates to a modifying reagent for a protein having a β-sheet structure, a modified protein, an article and a sensor device, and a method for modifying a protein.

### Description of the Related Art

A protein has characteristics such as biocompatibility, low toxicity, hydrophilicity, and low environmental impact. In addition, a protein exhibits various properties depending on the combination and sequence of amino acids and can be used as materials and ingredients in a wide variety of fields such as food and medicine. Among them, a protein having a β-sheet structure is characterized by high rigidity and easy aggregation. Therefore, a protein having a β-sheet structure is expected to be used as scaffold materials for environmental use, pharmaceutical use, regenerative medicine, optical devices, and electronic devices.

On the other hand, depending on the amino acid sequence, a protein may have few sites that can be chemically modified, and covalent chemical modification thereto may be difficult.

Fibroin contained in a silk fiber is listed as a protein having a ß-sheet structure. Fibroin has a hydrophobic crystal region that is rich in glycine, an amino acid with no side chain, and alanine, an amino acid with a small side chain. Because the crystal region adopts the β-sheet structure, the strong interaction works, and the rigid fiber is formed in fibroin. Japanese Patent Laid-Open No. 2022-529644 discloses a function imparted fibroin by chemical modification. In Japanese Patent Laid-Open No. 2022-529644, a function imparted fibroin is produced by covalently bonding molecules having properties desired to be imparted to fibroin using a chemical reaction. Japanese Patent No. 6362878 and Biotechnol Lett 2011, 33, 1069-1073 disclose a fibroin functionally imparted by using a peptide adsorbing to a fibroin molecule. The functionalized fibroin is simply prepared by adsorbing a peptide connected with a molecule having a desired property via a non-covalent bond to the fibroin.

### SUMMARY

Fibroin has few sites in its amino acid sequence that can be chemically modified. Therefore, it may be difficult to impart many desired molecules to fibroin by chemical modification. Further, the chemical modification requires a long reaction time, and the operation of the removal step of the reaction reagent is complicated.

There was a problem that the above-mentioned peptide easily caused steric hindrance in the function imparting to the fibroin due to the large molecular size. Therefore, the purpose of this disclosure is to modify a protein having a β-sheet structure by using a modifying reagent with a small binding site.

An aromatic compound, such as a dye, is small in molecular size and can noncovalently adsorb more aromatic compounds to a protein, by the similar mechanism as in CBB staining in gel electrophoresis. Accordingly, the inventors have focused on imparting a group containing an aromatic ring that adsorbs with a protein having a β-sheet structure to a molecular structure having a desired characteristic and have come to the present disclosure. That is, the present disclosure provides a modifying reagent for a protein having a β-sheet structure comprising a structure derived from a biomolecule and a group containing an aromatic ring, wherein the group containing the aromatic ring has at least one selected from a group consisting of a benzothiazole group, a benzoxazole group, a benzimidazole group, and a naphthylazo group, each of which may have a substituent, and wherein the structure derived from the biomolecule includes a structure derived from one selected from a group consisting of an enzyme, an antibody, an antigen, a peptide, a polynucleotide, an oligonucleotide, a ligand, an enzyme substrate, a biotin, and a catecholamine.

Features of the present disclosure will become apparent from the following description of embodiments.

### DESCRIPTION OF THE EMBODIMENTS

The present disclosure provides, as a first embodiment, a modifying reagent for a protein having a β-sheet structure, and comprising a structure derived from a biomolecule and a group containing an aromatic ring, wherein the group containing the aromatic ring has at least one selected from the group consisting of a benzothiazole group, a benzoxazole group, a benzimidazole group, and a naphthylazo group, each of which may have a substituent, and wherein the structure derived from the biomolecule includes a structure derived from any of the groups consisting of an enzyme, an antibody, an antigen, a peptide, a polynucleotide, an oligonucleotide, a ligand, an enzyme substrate, a biotin, and a catecholamine.

### Modifying Reagent for Protein Having β-Sheet Structure

A modifying reagent for a protein having a β-sheet structure according to the present embodiment may be provided that a group containing an aromatic ring linked through a covalent bond, an ionic bond, a hydrogen bond, or an intermolecular force, is bonded to a structure derived from a biomolecule (hereinafter also referred to as biomolecular structure). Since the biomolecular structure and the group containing the aromatic ring exist stably without dissociation, it is preferable that they are bonded through a covalent bond. The biomolecular structure may be directly bonded to a group containing an aromatic ring, and the bonding site may be bonded to the biomolecular structure using an aromatic compound having a functional group serving as a bonding site to the biomolecular structure. Alternatively, a group containing an aromatic ring may be linked to a biomolecular structure through a linker having a backbone of a hydrocarbon, oligoethylene glycol, polyethylene glycol, polypropylene glycol, or the like. The bonds used between a biomolecular structure and a group containing an aromatic ring, a biomolecular structure and a linker, and a linker and a group containing an aromatic ring may use, but are not limited to, amide bonds, ester bonds, ether bonds, thioether bonds, disulfide bonds, imine bonds, oxime bonds, hydrazide bonds, phosphate ester bonds, and 1,2,3-triazole bonds. Also, if these molecular structures can be formed, the method is not limited.

### Group Containing an Aromatic Ring

The group containing the aromatic ring in the present embodiment comprises at least one of a benzothiazole group, a benzoxazole group, a benzimidazole group, and a naphthylazo group. Specific examples of groups containing aromatic rings are shown in formulae (1) to (18) below. These groups have adsorptivity to proteins having a β structure. However, the group containing the aromatic ring is not limited to the following formulae (1) to (18) as long as it has absorptivity to a protein having a β structure.

In the following formula, X is either S, O, or NH.

S, O, and NH are all divalent, and S, O, and N have close atomic numbers, suggesting that they are likely to adopt similar molecular structures when replaced.

R¹ to R³ independently represent hydrogen atoms, substituted or unsubstituted aliphatic hydrocarbon groups having 1 to 4 carbon atoms, substituted or unsubstituted aryl or aralkyl groups having 6 to 10 carbon atoms, or alkoxy groups. Symbol * indicates a position of a bonding site.

The group containing the aromatic ring may be linked to a biomolecular structure through a linker having a backbone such as a hydrocarbon, oligoethylene glycol, polyethylene glycol, polypropylene glycol, or the like, or may be linked without a linker. Amide, ester, ether, thioether, disulfide, imine, oxime, hydrazide, phosphate, and 1,2,3-triazole bonds may be used between the biomolecular structure and the group containing the aromatic ring, or between the linker and the group containing the aromatic ring but are not limited thereto.

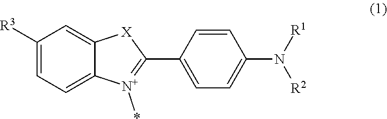

As preferred examples of formula (1), formulae (1-1) to (1-3) can be given.

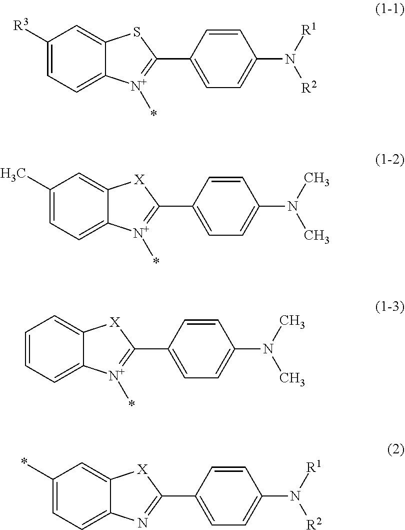

As preferred examples of formula (2), formulae (2-1) to (2-2) can be given.

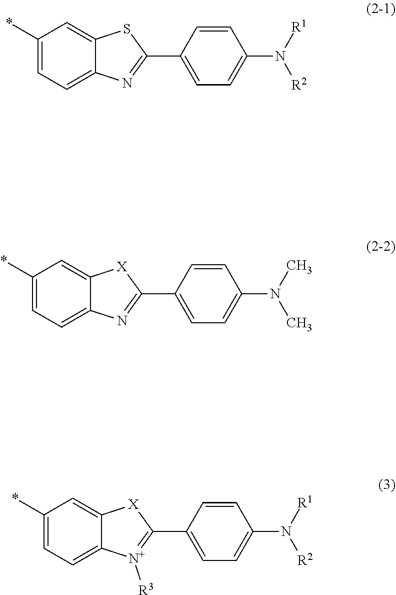

As preferred examples of formula (3), formulae (3-1) to (3-2) can be given.

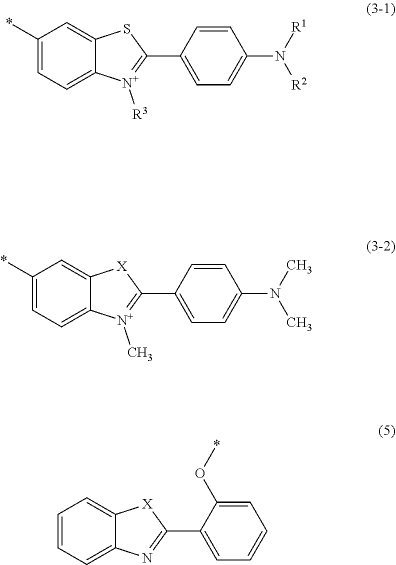

As preferred example of formula (5), formula (5-1) can be given.

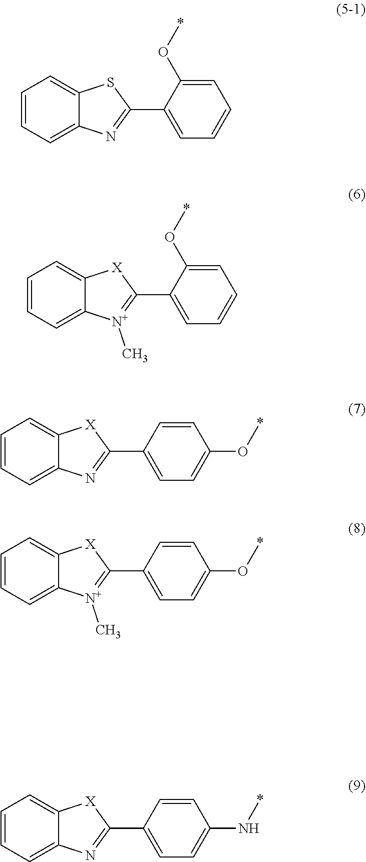

As preferred example of formula (9), formula (9-1) can be given.

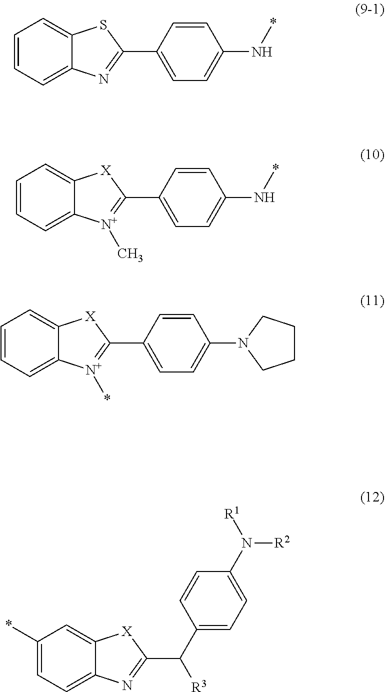

As preferred example of formula (12), formula (12-1) can be given.

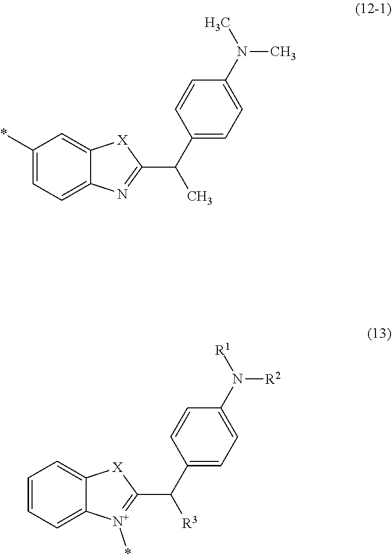

As preferred example of formula (13), formula (13-1) can be given.

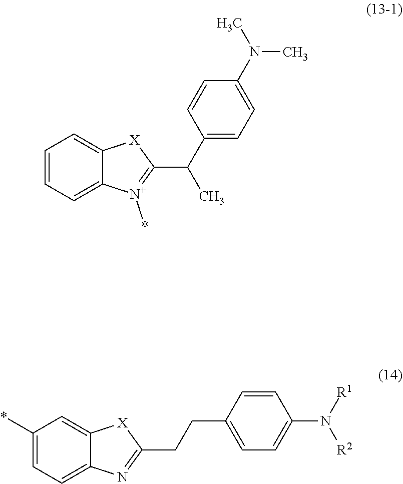

As preferred example of formula (14), formula (14-1) can be given.

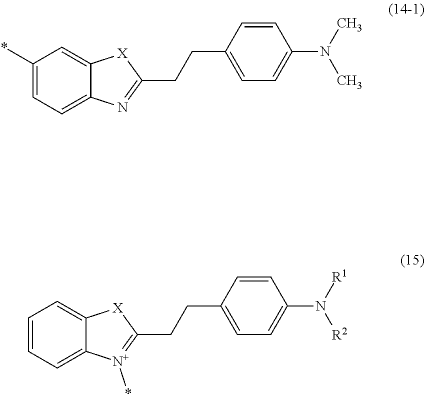

As preferred example of formula (15), formula (15-1) can be given.

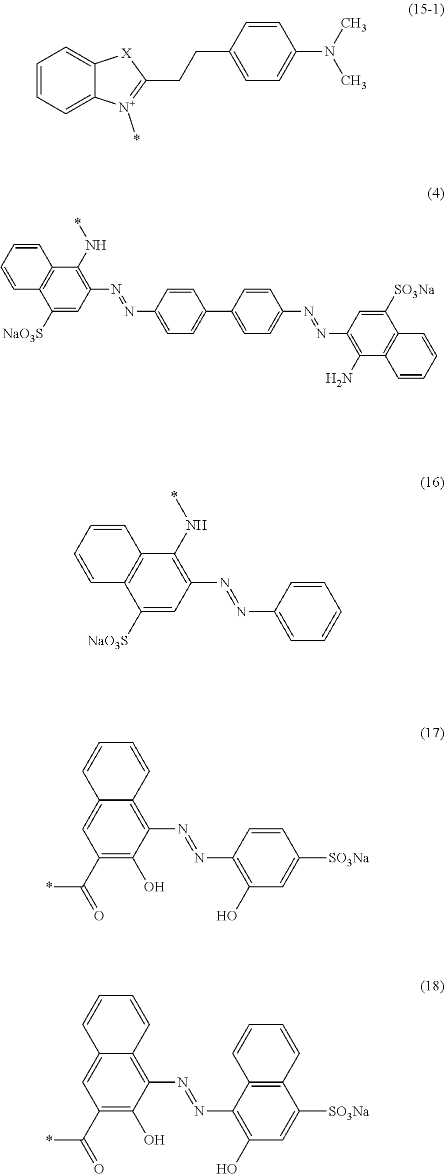

Among them, a monovalent group represented by formula (1-1), formula (2-1), formula (3-1), formula (4), formula (5-1), and formula (9-1) can be given as a group containing a particularly preferable aromatic ring.

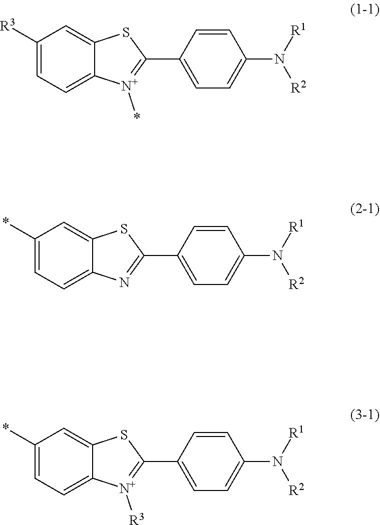

(In the formula (1-1), (2-1), (3-1), R¹ and R² independently represent a hydrogen atom, a substituted or unsubstituted aliphatic hydrocarbon group having 1 to 4 carbon atoms, or an alkoxy group, R³ represents a hydrogen, a substituted or unsubstituted aliphatic hydrocarbon group having 1 to 4 carbon atoms, or an alkoxy group, and symbol * indicates a bonding site.)

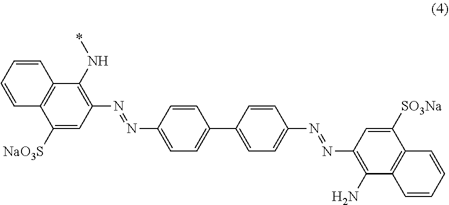

(In the above formula (4), symbol * indicates a bonding site.)

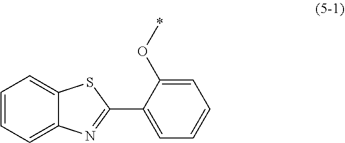 [SMILES: *Oc1ccccc1-c1nc2ccccc2s1]

(In the above formula (5-1), symbol * indicates a bonding site.)

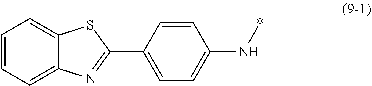 [SMILES: *Nc1ccc(-c2nc3ccccc3s2)cc1]

(In the above formula (9-1), symbol * indicates a bonding site.)

### Biomolecular Structure

The biomolecular structure in this embodiment includes a structure derived from one selected from a group consisting of an enzyme, an antibody, an antigen, a peptide, a polynucleotide, an oligonucleotide, a ligand, an enzyme substrate, a biotin, and a catecholamine.

If the enzyme, the antibody, the antigen, the peptide, or the ligand is a protein, it may be extracted from a natural protein, may be produced by genetic modification, may be a part of these, may be a combination, such as a chimera, and may include an amino acid sequence other than that derived from them. Examples of such amino acid sequences include repeating histidine structures, “FLAG” (registered trademark) sequences, polyglycine sequences, and the like.

Enzymes include, but are not limited to, those that catalyze redox reactions, transfer reactions, hydrolysis reactions, dissociation reactions, isomerization reactions, and further specific examples include horseradish peroxidase (HRP), alkaline phosphatase (ALP), β-galactosidase, and glucose oxidase with a potential change.

The antibody includes an antibody, a Fab, a single-chain antibody, a recombinant antibody, a chimeric antibody, and the like. There is no limitation to the origin of the antibody, and antibodies such as rabbit, rat, mouse, camel, and human can be used. The specificity of the antibody is not limited, for example, antigens include allergens, bacteria, viruses, cells, cell membrane components, cancer markers, various disease markers, antibodies, blood-derived substances, food-derived substances, natural product-derived substances, and antibodies recognizing any low-molecular-weight compound.

Anything recognized by an antibody can be an antigen, without particular limitation, including allergens, bacteria, viruses, cells, cell membrane components, cancer markers, various disease markers, antibodies, blood derived substances, food derived substances, natural product derived substances, and any low molecular weight compound.

The peptide includes, but is not limited to, for example, the histidine structure described above, the FLAG sequence, the polyglycine sequence, and the like.

Polynucleotides and oligonucleotides include, without particular limitation, DNA, RNA, cDNA, parts or fragments of them, synthetic nucleic acids, primers, probes, etc. from bacteria, viruses, cells, etc. Examples of ligands include any that bind a specific target, such as various receptors or membrane proteins that recognize cytokines, hormones, neurotransmitters, signal transducers, membrane proteins, etc.

As an enzyme substrate, any substance that is catalyzed by an enzyme is used without limitation, and a substance that produces color development, light emission, color change, or potential change by an enzyme reaction can be mentioned as a suitable example, for example, a fluorescent substance itself such as a para-nitrophenol derivative or a coumarin derivative, or a peptide labeled with a fluorescent substance or a chromogenic dye.

By adsorbing a modifying reagent having a group containing an aromatic ring to a biomolecular structure to a protein having a β-sheet structure, functions such as enzyme catalytic ability, antigen-antibody reactivity, immunoreactivity, cell adhesiveness, cell proliferation, gene adsorptivity, protein adsorptivity, enzyme reactivity, material immobilization ability and material adhesiveness can be imparted, or performance can be promoted.

The modifying reagent may be bonded to a group containing an aromatic ring through a functional group of the biomolecular structure. Alternatively, a bonding functional group may be introduced by chemical or genetic manipulation. It may be bonded to a group containing an aromatic ring through a linker having a backbone of a hydrocarbon, oligoethylene glycol, polyethylene glycol, polypropylene glycol, or the like. The bonds used between a biomolecular structure and a group containing an aromatic ring or between a biomolecular structure and a linker may include, but are not limited to, amide, ester, ether, thioether, disulfide, imine, oxime, hydrazide, phosphate, and 1,2,3-triazole bonds. If these bonds can be formed, the method is not limited.

### Protein Having β-Sheet Structure

The β-sheet structure is the secondary structure of a protein and is a fold structure composed of several polypeptides joined in parallel by hydrogen bonds. However, the state of secondary structure formation depends on the sequence of the protein and the environment around the molecule. Proteins having a β-sheet structure may be globular, filamentous, membrane, or glycoprotein, and include specifically fibroin, streptavidin, immunoglobulin, ovalbumin, ribonuclease, thioredoxin, DNA polymerase, glutaminase, ferredoxin, frataxin, green fluorescent protein, lysozyme, amyloid β, and α-synuclein. They may also be proteins having the β-sheet structure described in the Protein Conformation Classification Database (SCOP/SCOP2) or may be proteins exhibiting spectra derived from the β-sheet structures by infrared spectroscopy or circular dichroism analysis. They may also be treated to increase the β-sheet site in the presence of a crosslinking agent. The β-sheet structure may be amorphous or crystalline and may be subjected to a process of crystallization. In FT-IR analysis using the ATR method, a protein for which the ratio of β-sheet structure per protein molecule is calculated to be 1% or more can be defined as a protein having β-sheet structure. Specifically, it can be analyzed by the methods described in Nature Materials 2020, 19, 102-108. β-sheet structures are not only found within a single protein molecule, but are also formed by intermolecular association, as in fibroin. Since a β-sheet structure can be formed as long as it has an amino acid sequence to be a β-strand, the presence of a β-sheet structure of 1% or more per protein molecule can sufficiently adsorb the modifying reagent. The larger the proportion of the β-sheet structure, the easier the aromatic ring is to adsorb to the protein and the higher the modification efficiency, which is preferable in terms of modification.

### Fibroin

Fibroin, a preferred example of a protein having a β-sheet structure, will be described.

Fibroins are filamentous proteins derived from organisms in the orders Lepidoptera, Hymenoptera, or Araneae, and may have been obtained by transgenic techniques. Fibroin derived from the cocoon of the domestic silkworm is preferable from the viewpoint of easy availability of raw material.

The fibroin used in the present embodiment can be obtained from cocoons of silkworms, cocoon yarn, a cocoon yarn processed product (silk yarn, etc.), and residual yarn of the cocoon yarn processed product. The fibroin can be obtained from these raw materials by removing sericin and desalting it using a known scouring method.

The molecular weight of fibroin used in the present embodiment is not particularly limited, but the higher the molecular weight of the fibroin 10,000 or more, the higher the effect. Generally, the higher the molecular weight of the fibroin, the more likely it is to form a β-sheet structure, and the more likely the aqueous solution is to gelation or solidify. When the molecular weight is 10,000 or more, the mechanical properties of various structures formed from an aqueous solution are high, and it is preferable depending on the application of the structure.

### Modified Protein

The modifying reagent of a protein having a β-sheet structure according to the first embodiment of the present disclosure has a property of adsorbing to the β-sheet structure and is preferably used for modifying a protein having a β-sheet structure, for example, fibroin, with a biomolecular structure. That is, the present disclosure provides, as a second embodiment, a modifying reagent according to the first embodiment of the present disclosure and a protein having a β-sheet structure, wherein the modifying reagent is physisorbed with the protein through the β-sheet structure. The protein used in this embodiment can be any protein having a β-sheet structure. The modified protein of this embodiment may be in a solid state or a dissolved solution state. Proteins having a β-sheet structure are not particularly limited, but fibroin is preferred from the viewpoint of availability of raw materials.

The modified protein of this embodiment can be prepared by mixing a solution containing a modifying reagent of a protein having a β-sheet structure with a protein solution. The modified protein can also be prepared by adding a solution of a modifying reagent to a protein solid or a protein gel. Separation methods such as ultrafiltration and size exclusion chromatography can be used to separate excess modifying reagents or unreacted proteins from modified proteins. If the protein is solid and water-insoluble, the modified protein can be obtained by using common separation methods between solution and solid, such as filtration and centrifugation.

### Articles

The present disclosure provides, as a third embodiment, an article comprising a modified protein of the second embodiment. The article in the present embodiment may be, for example, a solid material having any one of the shapes of sheet, film, sponge, fiber, or nonwoven fabric, or may be in a form of a gel, or may be a molded body formed by a mold.

The manufacturing method of the article according to the present embodiment is not particularly limited and known methods can be utilized. For example, external stimuli that promote β-sheet formation can be used, specifically as the following. As a method for preparing a gel, for example, it is possible to use a method of changing the pH by hydrochloric acid or the like, a method of using a chemical substance by a gelation accelerator, a method of using a shear force by strong stirring or the like, and a method of applying an electric field. As a method for producing a sponge, a method using a porogen such as salt or sugar, a method of freeze-drying an aqueous solution and then annealing it by heat or a solvent or the like can be used. For example, a casting method or an electrospinning method can be used as a sheet manufacturing method. As a method for producing fibers, for example, a spinning method can be used. If necessary, general separation methods such as filtration and centrifugation can be used.

Using these fabrication methods, for example, films, sponges, fibers, nonwoven fabrics, gels, and molded bodies can be obtained from an aqueous solution of a protein having a β-sheet structure.

The article of this embodiment may be a sheet, a film, a sponge, a fiber, a nonwoven fabric, a gel, a molded body, or the like from an aqueous solution of the modified protein. Alternatively, the aqueous solution of the protein before modification may be made into a sheet, film, sponge, fiber, nonwoven fabric, gel, molded body, etc., and then modified with a modifying reagent of the protein having a β-sheet structure.

### Sensor Device

The present disclosure provides, as a fourth embodiment, a sensor device comprising an article containing a modified protein of a second embodiment of the present disclosure.

The sensor device according to the present embodiment is the article itself according to the third embodiment, the article arranged on the support, or the device on which the article is mounted on the sensing part, and is used for detecting, identifying, qualitatively analyzing, quantitatively analyzing, or otherwise obtaining information about a target substance. A signal is generated by the interaction or reaction between the article and the substance to be measured, and the substance can be detected by detecting the signal. A substance can also be detected by generating a signal by adding a detection material that generates a signal, such as a dye material, after the interaction or reaction.

The signal is, for example, a signal selected from the group consisting of electrical conductivity or resistance, current, potential, capacitance, absorbance, light transmittance, refractive index, fluorescence, phosphorescence, luminescence, color development, color change, and calorific value measured by calorimetry. The signal may be a color development pattern or color development intensity of the protein structure or detection material that is an article according to the third embodiment, or a fluorescence pattern or fluorescence intensity of a protein structure or detection material that is an article according to the third embodiment obtained by irradiation with ultraviolet or visible light using ultraviolet or LED lights.

For example, in the following embodiment, an enzyme is selected as a structure derived from a biomolecule of a modifying reagent of a protein having a β-sheet structure, and a sheet containing a protein modified by the enzyme is produced. With such a sheet, the presence of the substrate can be observed by a change in color and a change in absorbance. The sheet may be a sensor device as it is or may be a sensor device having the sheet as a sensing part.

For example, using a sheet on which HRP as an enzyme is adsorbed, the presence of hydrogen peroxide can be observed by a chromogenic reaction using a reagent combined 4-aminoantipyrine with a phenolic hydrogen donor, or a coloring reagent such as 3,3′-diaminobenzidine (DAB) or tetramethylbenzidine (TMB).

The coloring reagent may be contained in the sheet or used by adding it to the sheet. And glucose oxidase is an enzyme that produces hydrogen peroxide from glucose. That is, if the sheet on which glucose oxidase is adsorbed is used, the presence of glucose can be observed via a chromogenic reaction using the coloring reagent and HRP. In this case, the coloring reagent and HRP may be contained in the sheet or may be used by adding them to the sheet. Alternatively, the presence of glucose can also be observed using a sheet in which glucose oxidase and HRP are adsorbed.

By adopting an antibody as a structure derived from a biomolecule of a modifying reagent for a protein having a β-sheet structure, a sensor device for detecting an antigen to the antibody can be produced.

For example, by using a sheet adsorbed with an anti-human C-reactive protein antibody, the human C-reactive protein as an antigen can be captured by an antigen-antibody reaction, and the presence of the human C-reactive protein can be observed. An immune complex can be formed on the sheet by applying human C-reactive protein to the sheet on which the antibody is adsorbed, and then further applying a coloring material such as colloidal gold or a labeled anti-human C-reactive protein antibody modified with an enzyme such as HRP or alkaline phosphatase. An immune complex can also be formed by mixing the labeled antibodies with human C-reactive protein in advance and then acting on the sheet. The presence of an antigen, human C-reactive protein, can be observed by performing an enzyme-mediated chromogenic reaction on the immune complex, as in the Enzyme-Linked Immuno Sorbent Assay (ELISA) method or by measuring the degree of coloring of a coloring agent.

By adopting an antigen as a structure derived from a biomolecule of a modifier for a protein having a β-sheet structure, a sensor device for detecting an antibody to the antigen can be produced, and can be used for allergy testing, for example.

By adopting a polynucleotide or an oligonucleotide as a structure derived from a biomolecule of a modifying reagent for a protein having a β-sheet structure, a sensor device for detecting a nucleic acid sequence having complementarity can be produced, and when the polynucleotide has a function as an aptamer, a sensor device for detecting the target molecule can be produced.

A modified protein having avidin binding property can be obtained by adopting biotin as a structure derived from a biomolecule of a modifying reagent for a protein having a β-sheet structure. Further sites for recognizing antibodies, antigens, polynucleotides, oligonucleotides, enzymes, substrates, and other objects to be measured can be added to such modified proteins via avidin, and desired sensor devices can be produced.

### Method to Modify Proteins

The present disclosure provides, as a further embodiment, a method for modifying a protein comprising a step of mixing a modifying reagent for a protein having a β-sheet structure of the first embodiment and a protein having a β-sheet structure, and a method for modifying a protein comprising a step of coating a modifying reagent for a protein having a β-sheet structure of the first embodiment on a protein solid or a protein gel comprising a protein having a β-sheet structure.

### EXAMPLES

### Synthetic Example

Although the present disclosure will be more specifically described below with examples, the present disclosure is not limited by these examples unless the present disclosure goes beyond the scope thereof.

### (Manufacturing Example 1) Synthesis of Compound 1

Thioflavin T acid (manufactured by AAT Bioquest, Inc.) 2.8 mg, N-hydroxysuccinimide 7.7 mg, and 1-ethyl-3-[3-(dimethylamino) propyl] carbodiimide 16 mg were dissolved in 1.5 mL of dichloromethane (hereinafter DCM). After stirring overnight, the solvent was distilled off under reduced pressure and dissolved again in 0.25 mL of ethanol to obtain a solution of Thioflavin T acid N-hydroxysuccinimide ester. A solution of HRP in which 5 mg of horseradish peroxidase (manufactured by Sigma-Aldrich, hereinafter HRP) was dissolved in 1 mL of 0.1M boric acid buffer (pH8.5) was mixed with 0.02 mL of Thioflavin T acid N-hydroxysuccinimide ester solution, and the mixture was stirred at room temperature for 3 hours. The mixture was collected and dialyzed in PBS buffer (pH7.4) using a dialysis membrane with a fraction molecular weight of 2000. The filtrate was collected by filtration using a 0.22 μm filter to obtain an aqueous solution of Compound 1. UV-Vis spectrum measurements confirmed that the number of molecules of Thioflavin T acid bound to a single HRP molecule of Compound 1 was one molecule.

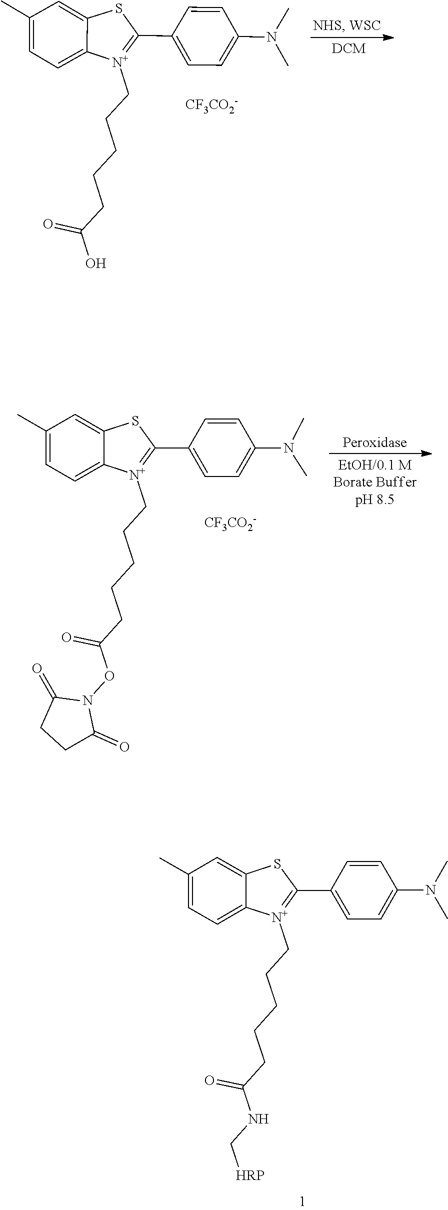

### (Manufacturing Example 2) Preparation of Fibroin Sheet Adsorbed With Compound 1

To 0.18 g of silk fibroin powder (manufactured by Sigma-Aldrich), 3.82 g of hexafluoro--2 propanol (HFIP) was added, and the mixture was stirred at room temperature using a mix rotor. Thereafter, filtration was performed using a filter (Millex-AP, Millipore) to obtain a 4.5 wt % fibroin solution. Fibroin solution (0.1 mL) was used to prepare and collect a sheet of fibroin fibers on an aluminum dish using an electrospinning device (NANON-03, MECC).

After methanol was added to the collected sheet, methanol was removed and dried overnight. The measurement of the weight of the fibroin sheet cut into 20 mm×40 mm size confirmed that it was 1.25 μg/mm² fibroin sheet. Fibroin sheets of 5 mm×5 mm size were cut and added to an aqueous solution of 0.3 mg/mL Compound 1 (0.2 mL) and allowed to stand for 10 minutes. Fibroin sheets were immersed in PBS buffer (1 mL, pH7.4) containing 0.05 wt % Tween20 for 15 minutes×2 and in PBS (1 mL, pH7.4) for 1 minute. The modified fibroin sheet was obtained by drying at room temperature.

### (Manufacturing Example 3) Preparation of HRP Adsorbed Fibroin Sheet

HRP adsorbed fibroin sheet was prepared in the same manner as above except that 0.3 mg/mL HRP aqueous solution was used instead of the aqueous solution of Compound 1.

### (Manufacturing Example 4) Synthesis of Compound 2

To 15 mL of N, N-dimethylformamide, 1.10 g of 2-(4-aminophenyl) benzothiazole (manufactured by Tokyo Chemical Industry Co., Ltd.) and 0.40 g of 3-bromopropionic acid (manufactured by Tokyo Chemical Industry Co., Ltd.) were added and refluxed under a nitrogen atmosphere for 4 hours. The solvent was evaporated at room temperature and purified by silica gel column chromatography to obtain 201 mg (26% yield) of a white solid of Compound 2. Identification was carried out by ¹H-NMR (manufactured by Bruker, Bruker Avance NEO 500, Resonance Frequency: 500 MHz) measurement equipment. ¹H-NMR (DMSO-d₆) (ppm): 12.30 (br, 1H), 8.06 (d, 1 H, J=7.8 Hz), 7.93 (d, 1 H, J=7.8 Hz), 7.86 (d, 2 H, J=9.0 Hz), 7.51-7.48 (m, 1H), 7.40-7.37 (m, 1H), 6.74 (d, 2 H, J=9.0 Hz), 6.53 (t, 1 H, J=5.5 Hz), 3.35 (m, 2H), 2.58 (m, 2H).

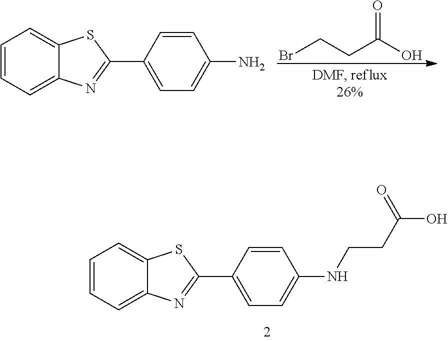 [SMILES: Nc1ccc(-c2nc3ccccc3s2)cc1 ; O=C(O)CCBr ; O=C(O)CCNc1ccc(-c2nc3ccccc3s2)cc1]

### (Manufacturing Example 5) Synthesis of Compound 3

Compound 2 1.7 mg, N-hydroxysuccinimide 5.5 mg, and 1-ethyl-3-[3-(dimethylamino)propyl] carbodiimide 15 mg were dissolved in 2.0 mL of DCM. After stirring for 1 hour, the solvent was distilled off under reduced pressure and dissolved again in 0.25 mL of ethanol to obtain a solution of Compound 2′. A solution of HRP dissolved with 5 mg of HRP in 1 mL of 0.1M boric acid buffer (pH8.5) and 0.1 mL of Compound 2′ solution were mixed and stirred at room temperature for 1.5 hours. The mixture was collected and dialyzed in PBS buffer (pH7.4) using a dialysis membrane with a fraction molecular weight of 2000. The filtrate was collected by filtration using a 0.22 μm filter to obtain an aqueous solution of Compound 3. The number of molecules of Compound 2 bound to a single HRP molecule of Compound 3 was confirmed to be 2.2 molecules by UV-Vis spectrum measurements.

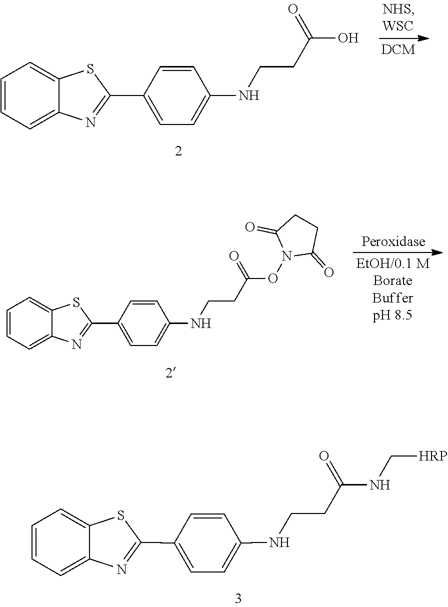

### (Manufacturing Example 6) Synthesis of Compound 4

To 20 mL of tetrahydrofuran, 1.01 g of 2-(2-hydroxyphenyl) benzothiazole (manufactured by Tokyo Chemical Industry Co., Ltd.), 0.67 g of 3-bromopropionic acid (manufactured by Tokyo Chemical Industry Co., Ltd.), and 1.52 g of potassium carbonate (manufactured by KISHIDA CHEMICAL CO., LTD.) were added, and refluxed under a nitrogen atmosphere for 3 hours. After returning to room temperature, 10 mL of methanol was added. The precipitate obtained by filtration was dried to obtain 57 mg of a white solid of Compound 4 (4% yield). Identification was carried out by ¹H-NMR (Bruker, Bruker Avance NEO 500, Resonance Frequency: 500 MHZ) measurement equipment. ¹H-NMR (DMSO-d6) (ppm): 12.52 (br, 1H), 8.50-8.48 (m, 1H), 8.13 (d, 1 H, J=8.0 Hz), 8.10 (d, 1 H, J=8.0 Hz), 7.61-7.56 (m, 2H), 7.50-7.46 (m, 1H), 7.37-7.36 (m, 1H), 7.23-7.20 (m, 1H), 4.52 (t, 2 H, J=6.0 Hz), 2.99 (t, 2 H, J=6.0 Hz).

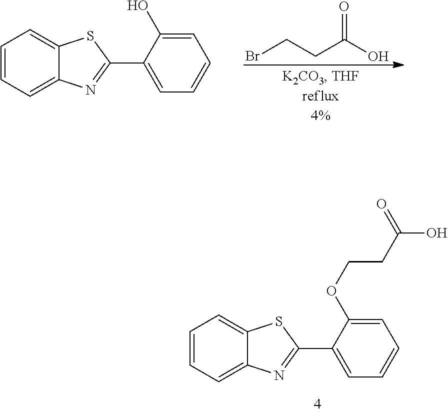 [SMILES: Oc1ccccc1-c1nc2ccccc2s1 ; O=C(O)CCBr ; O=C(O)CCOc1ccccc1-c1nc2ccccc2s1]

### (Manufacturing Example 7) Synthesis of Compound 5

Compound 4 1.7 mg, N-hydroxysuccinimide 5.8 mg, and 1-ethyl-3-[3-(dimethylamino) propyl] carbodiimide 17 mg were dissolved in 2.0 mL of DCM. After stirring for 1 hour, the solvent was distilled off under reduced pressure and dissolved again in 0.25 mL of ethanol to obtain a solution of Compound 4′. A solution of HRP dissolved with 5 mg of HRP in 1 mL of 0.1M boric acid buffer (pH8.5) and 0.03 mL of Compound 4′ solution were mixed and stirred at room temperature for 1.5 hours. The mixture was collected and dialyzed in PBS buffer (pH7.4) using a dialysis membrane with a fraction molecular weight of 2000. The filtrate was collected by filtration using a 0.22 μm filter to obtain an aqueous solution of Compound 5. UV-Vis spectral measurements confirmed that the number of molecules of Compound 4 bound to a single HRP molecule of Compound 5 was 2.6 molecules.

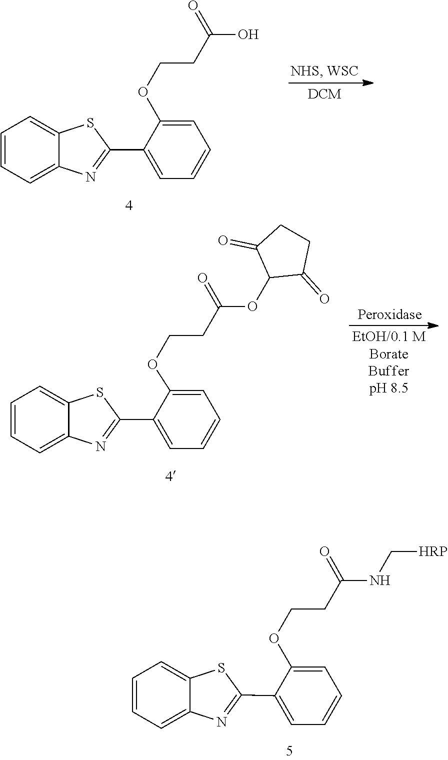

### (Manufacturing Example 8) Synthesis of Compound 6

Thioflavin T acid (Made by AAT Bioquest, Inc.) 1.2 mg, N-hydroxysuccinimide 4.4 mg, and 1-ethyl-3-[3-(dimethylamino) propyl] carbodiimide 4.8 mg were dissolved in 1.0 mL of DCM. After stirring overnight, the solvent was distilled off under reduced pressure and dissolved again in 0.05 mL of ethanol to obtain a Thioflavin T acid N-hydroxysuccinimide ester solution. A GOx solution obtained by dissolving 20 mg of glucose oxidase (manufactured by Sigma-Aldrich, G2133, hereinafter GOx) in 1 mL of 0.1M boric acid buffer (pH8.5) was mixed with 0.05 mL of Thioflavin T acid N-hydroxysuccinimide ester solution, and the mixture was stirred at room temperature for 2 hours. The mixture was collected and dialyzed in PBS buffer (pH7.4) using a dialysis membrane with a fraction molecular weight of 2000. The filtrate was collected by filtration using a 0.22 μm filter to obtain an aqueous solution of Compound 6. UV-Vis spectral measurements confirmed that the number of molecules of Thioflavin T acid bound to the GOx1 molecule of Compound 6 was 5.6 molecules.

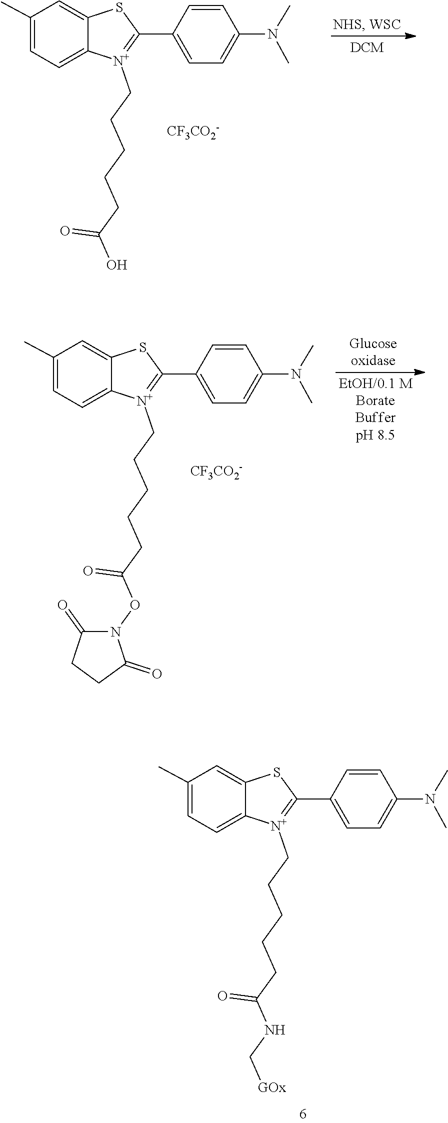

### (Manufacturing Example 9) Synthesis of Compound 7

Thioflavin T acid (Made by AAT Bioquest, Inc.) 1.2 mg, N-hydroxysuccinimide 4.4 mg, and 1-ethyl-3-[3-(dimethylamino) propyl] carbodiimide 4.8 mg were dissolved in 1.0 mL of DCM. After stirring overnight, the solvent was distilled off under reduced pressure and dissolved again in 0.05 mL of ethanol to obtain a Thioflavin T acid N-hydroxysuccinimide ester solution. 0.05 mL of mAb solution to which 0.05 mL of a 11.6 mg/mL anti-human C-reactive protein mouse monoclonal antibody solution (CRP-MCA, Product No. 47858000, manufactured by Oriental Yeast Industry Co., Ltd.) was added to 0.95 mL of 0.1M boric acid buffer (pH8.5), was mixed with 0.05 mL of Thioflavin T acid N-hydroxysuccinimide ester solution, and the mixture was stirred at room temperature for 2 hours. The mixture was collected and dialyzed in PBS buffer (pH7.4) using a dialysis membrane with a fraction molecular weight of 2000. The filtrate was collected by filtration using a 0.22 μm filter to obtain an aqueous solution of Compound 7. UV-Vis spectral measurements and BCA assays confirmed that the number of molecules of Thioflavin T acid bound to 1 molecule of Compound 7 mAb was 38 molecules.

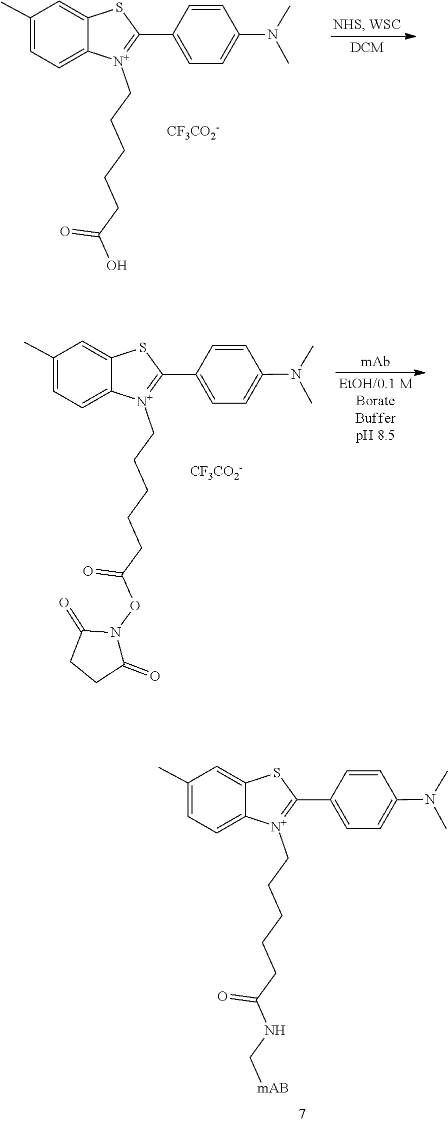

### (Manufacturing Example 10) Preparation of a Fibroin Sheet Adsorbed With Compound 3

A fibroin sheet adsorbed with Compound 3 was prepared in the same manner as Manufacturing Example 2 except that 0.3 mg/mL Compound 3 aqueous solution was used instead of the aqueous solution of Compound 1.

### (Manufacturing Example 11) Preparation of a Fibroin Sheet Adsorbed With Compound 5

A fibroin sheet adsorbed with Compound 5 was prepared in the same manner as Manufacturing Example 2 except that 0.3 mg/mL Compound 5 aqueous solution was used instead of the aqueous solution of Compound 1.

### (Manufacturing Example 12) Preparation of a Fibroin Sheet Adsorbed With Compound 6

A fibroin sheet adsorbed with Compound 6 was prepared in the same manner as Manufacturing Example 2 except that 0.3 mg/mL Compound 6 aqueous solution was used instead of the aqueous solution of Compound 1.

### (Manufacturing Example 13) Preparation of a Fibroin Sheet Adsorbed With GOx

A fibroin sheet adsorbed with GOx was prepared in the same manner as Manufacturing Example 2 except that 0.3 mg/mL GOx aqueous solution was used instead of the aqueous solution of Compound 1.

### (Manufacturing Example 14) Preparation of a Fibroin Sheet Adsorbed With Compound 7

A fibroin sheet adsorbed with Compound 7 was prepared in the same manner as Manufacturing Example 2 except that 0.02 mg/mL Compound 7 aqueous solution was used instead of the aqueous solution of Compound 1.

### (Manufacturing Example 15) Preparation of a Fibroin Sheet Adsorbed With mAb

A fibroin sheet adsorbed with mAb was prepared in the same manner as Manufacturing Example 2 except that 0.02 mg/mL mAb aqueous solution was used instead of the aqueous solution of Compound 1.

In the same method as in Manufacturing Example 2, an aqueous solution of Compound 1 was changed to a 0.02 mg/mL mAb aqueous solution to prepare.

### (Evaluation of Adsorptivity of Groups Containing Aromatic Rings to Proteins Having β-Sheet Structures)

Methanol solutions (0.02 mM, 0.15 mL) of Thioflavin T acid, Compound 2, and Compound 4, 7-(diethylamino) coumarin-3 carboxylic acid, compounds having groups containing aromatic rings, were prepared, and added to 10 mg of silk fibroin powder (manufactured by Sigma-Aldrich). After distillation of methanol by air-drying, 0.15 mL of water was added. After stirring for 5 minutes, the supernatant was collected. The adsorption rate of groups containing aromatic rings to fibroin was calculated from the concentration of groups containing aromatic rings in the collected supernatant liquid and the change in concentration by absorbance measurement, the adsorptivity of groups containing aromatic rings to proteins having β-sheet structures was evaluated using the following criteria.

“Evaluation A” and “Evaluation B” indicate the level at which groups containing aromatic rings adsorb strongly to proteins having a β-sheet structure, and “Evaluation C” indicates the low adsorptivity of groups containing aromatic rings.

### (Criteria)

A: 90% or more
B: 60% or more but less than 90%
C: Less than 60%

TABLE 1

| Compound having | | Com- | Com- | 7-(Diethylamino) |
|---|---|---|---|---|
| group containing | Thioflavin | pound | pound | Coumarin-3 |
| aromatic ring | T acid | 2 | 4 | Carboxylic Acid |
| Adsorption rate to | 91 | 79 | 71 | 55 |
| fibroin (%) | | | | |
| Adsorptivity | A | B | B | C |

### Example 1

To the fibroin sheet modified with Compound 1 obtained in Manufacturing Example 2, a staining solution (1.5 μL) of the peroxidase staining DAB kit (Brown Stain) (manufactured by Nacalai Tesque) was added dropwise and allowed to stand for 5 minutes in a wet box. The sheet was then washed with pure water (10 mL). After drying the sheet, the sheet was read by a scanner to acquire image data. The obtained image data were measured for color intensity using ImageJ software. Five sheets were made, and the same evaluation was carried out, and the average value of the color intensity (Color Intensity, hereinafter CI value) was calculated. The calculated CI value was 59.8.

### Example 2

The measurement was carried out in the same manner as in Example 1, except for replacing a fibroin sheet with a fibroin sheet to which Compound 3 was adsorbed. The calculated CI value was 58.1.

### Example 3

The measurement was carried out in the same manner as in Example 1, except for replacing a fibroin sheet with a fibroin sheet to which Compound 5 was adsorbed. The calculated CI value was 54.4.

### Comparative Example 1

The measurement was carried out in the same manner as in Example 1, except for replacing a sheet with a fibroin sheet to which a HRP prepared in Manufacturing Example 3 was absorbed. The calculated CI value was 39.5.

Thus, when the modifying reagent according to the embodiment of the present disclosure is used, it has been found that a modified fibroin containing a larger amount of the structure derived from HRP can be formed.

### Example 4

1.5 μL of phosphate buffered saline (pH7.4) containing 10 mM glucose, 1000 U/mL HRP, 3.7 mM 4-aminoantipyrine, 6.4 mM sodium 3-[ethyl(m-tolyl) amino]-2 hydroxy-1 propanesulfonate was added dropwise to a fibroin sheet modified with Compound 6 obtained in Manufacturing Example 12 and allowed to stand in a wet box for 5 minutes. The sheet was then washed with 10 mL of pure water. After drying the sheet, the sheet was read by a scanner to obtain image data. The resulting image data were subjected to color intensity measurement using ImageJ software. Five sheets were prepared and subjected to the same evaluation, and the average value of color intensity (Color Intensity, hereinafter CI value) was calculated. The calculated CI value was 54.5.

### Comparative Example 2

The measurement was carried out in the same method as in Example 1, replacing a sheet with a fibroin sheet with adsorbed GOx prepared in Manufacturing Example 13. The calculated CI value was 30.8.

Thus, when the modifying reagent according to the embodiment of the present disclosure is used, it has been found that a modified fibroin containing more structures derived from GOx can be formed.

### Example 5

1.5 μL of phosphate-buffered saline (pH7.4) containing 100 nM alkaline phosphatase-modified mAb, 100 nM human C-reactive protein, and 1.0 wt % bovine serum albumin was added dropwise to a fibroin sheet modified with Compound 7 obtained in Manufacturing Example 14 and allowed to stand in a wet box for 5 minutes. The sheet was then washed with phosphate-buffered saline (10 mL). 1.0 μL of BCIP/NBT solution (manufactured by Fujifilm Wako Pure Chemical Corporation) was added dropwise and allowed to stand in a wet box for 10 minutes. The sheet was then washed with pure water (10 mL). After drying the sheet, the sheet was read by a scanner to acquire image data. The obtained image data were measured for color intensity using ImageJ software. Five sheets were made, and the same evaluation was carried out, and the average value of the color intensity (Color Intensity, hereinafter CI value) was calculated. The calculated CI value was 39.5.

### Comparative Example 3

The measurement was carried out in the same method as in Example 1, replacing a sheet with a fibroin sheet to which a mAb prepared in Manufacturing Example 15 was adsorbed. The calculated CI value was 23.4.

Thus, when the modifying reagent according to the embodiment of the present disclosure is used, it has been found that a modified fibroin containing more structures derived from antibodies can be formed.

A function imparting of the protein became possible using a protein modifying reagent having the β-sheet structure comprising a biomolecular structure and a group containing an aromatic ring.

While the present disclosure has been described with reference to embodiments, it is to be understood that the present disclosure is not limited to the disclosed embodiments. The scope of the following claims is to be accorded the broadest interpretation so as to encompass all such modifications and equivalent structures and functions.

1. A modifying reagent for a protein having a β-sheet structure, comprising a structure derived from a biomolecule and a group containing an aromatic ring,
wherein the group containing the aromatic ring may has at least one selected from the group consisting of a benzothiazole group, a benzoxazole group, a benzimidazole group, and a naphthylazo group, each may have a substituent,
the structure derived from the biomolecule includes a structure derived from one selected from a group consisting of an enzyme, an antibody, an antigen, a peptide, a polynucleotide, an oligonucleotide, a ligand, an enzyme substrate, a biotin, and a catecholamine.

2. The modifying reagent of claim 1, wherein the group containing the aromatic ring comprises at least one of a benzothiazole group and a naphthylazo group.

3. The modifying reagents according to claim 1, wherein the group containing the aromatic ring is any one of monovalent groups represented by formula (1-1), formula (2-1), formula (3-1), formula (4), formula (5-1), and formula (9-1) 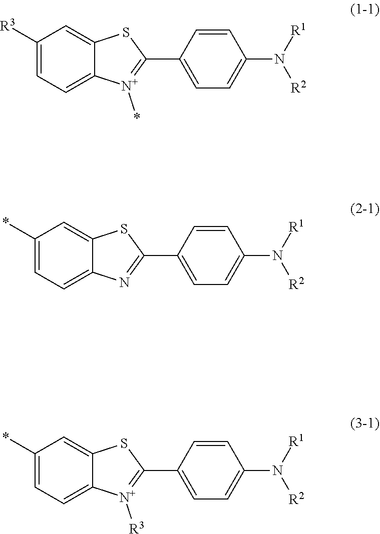
in the above formula (1-1), formula (2-1), formula (3-1),
R¹ to R³ independently represent a hydrogen atom, a substituted or unsubstituted aliphatic hydrocarbon group having 1 to 4 carbon atoms,
a substituted or unsubstituted aryl, aralkyl, or alkoxy group having 6 to 10 carbon atoms, and
symbol * indicates a bonding site, 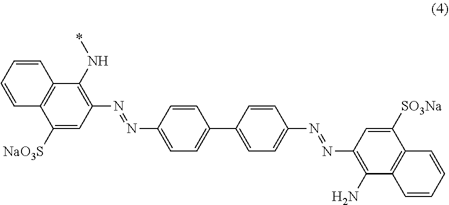
in the above formula (4), symbol * indicates a bonding site, 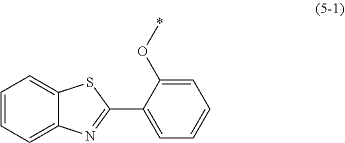 [SMILES: *Oc1ccccc1-c1nc2ccccc2s1]
in the above formula (5-1), symbol * indicates a bonding site, 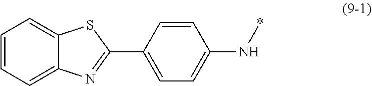 [SMILES: *Nc1ccc(-c2nc3ccccc3s2)cc1]
in the above formula (9-1), symbol * indicates a bonding site.

4. The modifying reagent of claim 1, wherein the structure derived from the biomolecule comprises a structure derived from anyone selected from a group consisting of an enzyme, an antibody, an antigen, an oligonucleotide, and a biotin.

5. A modified protein comprising the modifying reagent according to claim 1, and a protein having a β-sheet structure,
wherein the modifying reagent is physisorbed with the protein through the β-sheet structure.

6. An article according to claim 5 comprising the modified protein.

7. The article according to claim 6, wherein the article is in sheet form.

8. The article according to claim 6, wherein the article is in gel form.

9. The article according to 6, wherein the article is in a form of spongy.

10. The article according to 6, wherein the article is in a form of fibrous.

11. A sensor device comprising the modified protein according to claim 5.

12. A method for modifying a protein comprising a step of mixing the modifying reagent according to claim 1 with a protein having a β-sheet structure.

13. A method for modifying a protein comprising a step applying the modifying reagent according to claim 1 to a protein solid or protein gel comprising a protein having a β-sheet structure.
